(19) United States
(12) Patent Application Publication
(10) Pub. No.: US 2008/0312336 A1
(43) Pub. Date: Dec. 18, 2008

(54) Preventive and/or Therapeutic Agent for Diabetic Vascular Disorder and Respiratory Disorder

(76) Inventors: Keisuke Satoh, Tottori (JP); Motoaki Saito, Tottori (JP); Bang Luu, Strasbourg (FR); Masashi Yamada, Tokyo (JP); Hiroto Suzuki, Tokyo (JP)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/913,256

(22) PCT Filed: Nov. 24, 2005
(86) PCT No.: PCT/JP2005/021562

§ 371 Date: Jun. 23, 2008

(30) Foreign Application Priority Data
May 26, 2005 (JP) 2005-153990

(51) Int. Cl. A61K 31/122; A61P 11/00; A61P 9/00

(52) U.S. Cl. 514/690

(57) ABSTRACT

The present invention provides preventive and/or therapeutic agents for either one, or both, of a diabetic vascular disorder and a diabetic respiratory disorder comprising, as an active ingredient, a cyclohexenone long chain alcohol compound represented by the following formula (1):

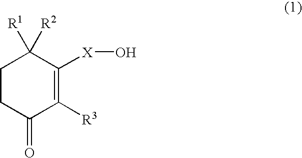

[wherein R¹, R², and R³ each represent a hydrogen atom or a methyl group, and X represents a linear or branched C10-C28 alkylene or alkenylene group].

Since the cyclohexenone long chain alcohol of formula (1) improves diabetic vascular smooth muscle contraction and bronchial smooth muscle contraction, it is useful as a preventive and/or therapeutic agent for a diabetic vascular disorder or a respiratory disorder.

### TECHNICAL FIELD

The present invention relates to nonpeptide low-molecular-weight compounds for preventing and/or treating vascular and tracheal smooth muscle dysfunctions caused by diabetes.

### BACKGROUND ART

In diabetes, ingested glucose is not easily taken up by cells such as muscle cells due to an impaired insulin action, causing an energy deficiency in these cells. Moreover, the impaired insulin action also inhibits the usage of proteins and lipids as well as sugars such as glucose. This triggers hyperglycemia and/or hyperlipemia, damaging blood vessels and nerves, and inducing various complications (Katsuo Kamata, “Diabetic Angiopathy and LDL Cholesterol”, an online article at the Hoshi University High-Tech Research Center website http://polaris.hoshi.ac jp/hitec/symp99/kamata.html (as searched on Feb. 10, 2005; publication date unknown, but described as presented on Nov. 6, 1999 at the Hoshi University High-Tech Research symposium, High-Tech Research Center Development Project, Ministry of Education).

With the increase of diabetic foot lesions such as foot ulcers and gangrenes, problems such as long-term hospitalization, foot amputation, and QOL decrease have become serious issues. In addition to diabetic complications such as neurological disorders and/or vascular disorders, weakened immunity due to a continuous hyperglycemic state is involved in the development of foot lesions (Non Patent Document 1).

Diabetic vascular disorders comprise microangiopathies such as diabetic retinopathy, diabetic nephropathy, and diabetic neuropathy, and macroangiopathies such as cerebrovascular disorders, ischemic heart disease, and diabetic gangrenes (Makoto Utsumi, “Diabetes: Diabetic Complications”, Utsumi Internal Medicine Clinic, an online article: http://www.furano.ne.jp/utsumi/dm/complications.htm (as searched on Feb. 10, 2005; publication date unknown)). Moreover, diabetic patients have a high carotid artery thickening, the associated risk factors being age, high blood pressure, lipid abnormalities and the like (Non Patent Document 2).

Experimental diabetic rats have been reported to have decreased tracheal smooth muscle functions (Non Patent Document 3, Non Patent Document 4, and Non Patent Document 5), raising concerns of respiratory difficulties due to diabetes.

The effects of γ-aminobutyric acid (GABA), and GABA receptor agonist and antagonist on tracheal contraction were investigated using streptozotocin-induced diabetic rats and normal rats. Tracheal contraction induced by electrical stimulation was inhibited by GABA and GABA β receptor agonist. The contraction-inhibitory effect of GABA was significantly high in normal rats, which suggested that the action mechanism of GABA includes inhibition of acetylcholine release through GABA β receptors. The tracheal contraction-inhibitory effect of GABA was found to be impaired in diabetes (Non Patent Document 3).

Rats were intravenously administered with 65 mg/kg of streptozotocin, their tracheas were isolated six weeks after the administration, and tracheal specimens were prepared to compare with those from control animals. The phosphodiesterase (PDE) III inhibitor aminone, the PDE IV inhibitor rolipram, and the nonselective PDE inhibitor theophylline all demonstrated concentration-dependent relaxation of specimens contracted with carbachol (10⁻⁶ mol/l). In control animal specimens, the effect of rolipram was observed at the lowest concentration. The diabetic rat specimens showed higher response to amrinone than those from control animals, but there was no significant difference in responses to theophylline and rolipram (Non Patent Document 4).

Effect of nitric oxide (NO) on acetylcholine-induced contraction and electrical stimulation-induced contraction was investigated using tracheal muscles isolated from streptozotocin-induced diabetic rats. Acetylcholine-induced contraction was not affected by the NO synthase blocker, N_{G}-nitro-L-arginine-methylester (L-NAME). Electrical stimulation-induced contraction was enhanced in diabetic rats. L-NAME enhanced the response in normal rats, but not in diabetic rats. These results suggested that the production or release of endogenous NO might be impaired in diabetic rats (Non Patent Document 5).

Non Patent Document 1: Kazunori Koyama, Diabetic Foot Lesions—for preventing diabetic foot gangrene—, Infection Prevention, 14(4), pp. 1-8 (2004)

### Non Patent Document 2: Yoshiki Nishizawa, Angiopathy in Diabetic Patients, Medical Consultation & New Remedies, 41(5), pp. 372 (2004)

Non Patent Document 3: Ozdem S S, Sadan G, Usta C, Tasatargil A, Effect of experimental diabetes on GABA-mediated inhibition of neurally induced contractions in rat isolated trachea: role of nitric oxide, Clin. Exp. Pharmacol. Physiol., 27(4), pp. 299-305 (2000)

### Non Patent Document 4: Usta C, Sadan G, A Comparison of the Effects of Selective III, IV and Nonselective Phosphodiesterase Inhibitors on Isolated Tracheal Preparations in Streptozotocin-Diabetic Rats, Pharmacology, 60(1), pp. 9-12 (2000)

Non Patent Document 5: Ozdem S S, Sadan G, Usta C, Tasatargil A, The effect of experimental diabetes on cholinergic neurotransmission in rat trachea: role of nitric oxide, Eur. J. Pharmacol., 387(3), pp. 321-327 (2000)
Patent Document 1: Japanese Patent Application Kokai Publication No. (JP-A) 2000-297034 (unexamined, published Japanese patent application)

### Patent Document 2: JP-A 2002-241270

### Patent Document 3: JP-A 2002-241271

### DISCLOSURE OF THE INVENTION

### Problems to be Solved by the Invention

An objective of the present invention is to provide nonpeptide low-molecular-weight compounds which prevent and/or treat vascular or tracheal smooth muscle dysfunctions caused by diabetes.

### Means for Solving the Problems

It has already been reported that a cyclohexenone long chain alcohol has a neurotrophic function which promotes neuron survival and neurite extension (Gonzalez de Aguilar J L, Girlanda-Junges C, Coowar D, Duportail G, Loeffler J P, Luu B, Neurotrophic activity of 2,4,4-trimethyl-3-(15-hydroxypentadecyl)-2-cyclohexen-1-one in cultured central nervous system neurons, Brain Res., 920(1-2), pp. 65-73 (2001), and JP-A 2000-297034). This compound has also been reported to be useful as a therapeutic agent for diabetic neuropathy (JP-A 2002-241270) and dysuria (JP-A 2002-241271).

This time, the present inventors discovered that cyclohexenone long chain alcohol compounds represented by formula (I) improve vascular and tracheal smooth muscle dysfunctions caused by diabetes, and completed the present invention.

That is, an objective of the present invention is to provide methods for preventing/treating at least one disorder selected from the group consisting of the diabetic vascular disorders and diabetic respiratory disorders mentioned below.

[1] A method for either one, or both, of preventing and treating at least one disorder selected from the group consisting of diabetic vascular disorders and diabetic respiratory disorders, comprising the step of administering a cyclohexenone long chain alcohol compound represented by the following formula (1):

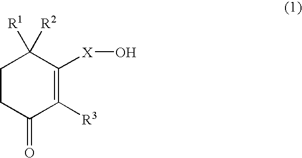

[wherein R¹, R², and R³ each represent a hydrogen atom or a methyl group, and X represents a linear or branched C10-C28 alkylene or alkenylene group].
[2] The method of [1], wherein R¹ is a methyl group and X is a linear C10-C28 alkylene group.
[3] The method of [2], wherein R² is a methyl group.
[4] The method of either one of [2] and [3], wherein R³ is a methyl group.
[5] The method of [1], wherein R¹ and R² are hydrogen atoms.
[6] The method of [1], wherein the cyclohexenone long chain alcohol compound is selected from the group consisting of the following compounds:
3-(10-hydroxydecyl)-2-cyclohexen-1-one (3-(10-hydroxydecyl)-2-cyclohexenone);
3-(11-hydroxyundecyl)-2-cyclohexen-1-one (3-(11-hydroxyundecyl)-2-cyclohexenone);
3-(12-hydroxydodecyl)-2-cyclohexen-1-one (3-(12-hydroxydodecyl)-2-cyclohexenone);
3-(13-hydroxytridecyl)-2-cyclohexen-1-one (3-(13-hydroxytridecyl)-2-cyclohexenone);
3-(14-hydroxytetradecyl)-2-cyclohexen-1-one (3-(14-hydroxytetradecyl)-2-cyclohexenone);
3-(10-hydroxydecyl)-4-methyl-2-cyclohexen-1-one (3-(10-hydroxydecyl)-4-methyl-2-cyclohexenone);
3-(11-hydroxyundecyl)-4-methyl-2-cyclohexen-1-one (3-(11-hydroxyundecyl)-4-methyl-2-cyclohexenone);
3-(12-hydroxydodecyl)-4-methyl-2-cyclohexen-1-one (3-(12-hydroxydodecyl)-4-methyl-2-cyclohexenone);
3-(13-hydroxytridecyl)-4-methyl-2-cyclohexen-1-one (3-(13-hydroxytridecyl)-4-methyl-2-cyclohexenone);
3-(14-hydroxytetradecyl)-4-methyl-2-cyclohexen-1-one (3-(14-hydroxytetradecyl)-4-methyl-2-cyclohexenone);
4,4-dimethyl-3-(10-hydroxydecyl)-2-cyclohexen-1-one (4,4-dimethyl-3-(10-hydroxydecyl)-2-cyclohexenone);
3-(11-hydroxyundecyl)-4,4-dimethyl-2-cyclohexen-1-one (3-(11-hydroxyundecyl)-4,4-dimethyl-2-cyclohexenone);
3-(12-hydroxydodecyl)-4,4-dimethyl-2-cyclohexen-1-one (3-(12-hydroxydodecyl)-4,4-dimethyl-2-cyclohexenone);
3-(13-hydroxytridecyl)-4,4-dimethyl-2-cyclohexen-1-one (3-(13-hydroxytridecyl)-4,4-dimethyl-2-cyclohexenone);
3-(14-hydroxytetradecyl)-4,4-dimethyl-2-cyclohexen-1-one (3-(14-hydroxytetradecyl)-4,4-dimethyl-2-cyclohexenone);
3-(10-hydroxydecyl)-2-methyl-2-cyclohexen-1-one (3-(10-hydroxydecyl)-2-methyl-2-cyclohexenone);
3-(11-hydroxyundecyl)-2-methyl-2-cyclohexen-1-one (3-(11-hydroxyundecyl)-2-methyl-2-cyclohexenone);
3-(12-hydroxydodecyl)-2-methyl-2-cyclohexen-1-one (3-(12-hydroxydodecyl)-2-methyl-2-cyclohexenone);
3-(13-hydroxytridecyl)-2-methyl-2-cyclohexen-1-one (3-(13-hydroxytridecyl)-2-methyl-2-cyclohexenone);
3-(14-hydroxytetradecyl)-2-methyl-2-cyclohexen-1-one (3-(14-hydroxytetradecyl)-2-methyl-2-cyclohexenone);
3-(12-hydroxydodecyl)-2,4,4-trimethyl-2-cyclohexen-1-one (3-(12-hydroxydodecyl)-2,4,4-trimethyl-2-cyclohexenone);
3-(13-hydroxytridecyl)-2,4,4-trimethyl-2-cyclohexen-1-one (3-(13-hydroxydodecyl)-2,4,4-trimethyl-2-cyclohexenone);
3-(14-hydroxytetradecyl)-2,4,4-trimethyl-2-cyclohexen-1-one (3-(14-hydroxytetradecyl)-2,4,4-trimethyl-2-cyclohexenone);
3-(15-hydroxypentadecyl)-2,4,4-trimethyl-2-cyclohexen-1-one (3-(15-hydroxypentadecyl)-2,4,4-trimethyl-2-cyclohexenone); and
3-(16-hydroxyhexadecyl)-2,4,4-trimethyl-2-cyclohexen-1-one (3-(16-hydroxyhexadecyl)-2,4,4-trimethyl-2-cyclohexenone).
   [7] The method of [1], wherein the disorder is a diabetic vascular disorder.
   [8] The method of [1], wherein the disorder is a diabetic respiratory disorder.
   [9] The method of [8], wherein the diabetic respiratory disorder is sleep apnea syndrome.
   [10] The method of [1], wherein the method is a therapeutic method.
   [11] A preventive and/or therapeutic agent for either one, or both, of a diabetic vascular disorder and a diabetic respiratory disorder, comprising, as an active ingredient, a cyclohexenone long chain alcohol compound represented by the following formula (1):

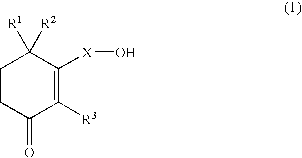

[wherein R¹, R², and R³ each represent a hydrogen atom or a methyl group, and X represents a linear or branched C10-C28 alkylene or alkenylene group].

Alternatively, the present invention relates to the use of a cyclohexenone long chain alcohol compound represented by formula (I) in the production of a preventive and/or therapeutic agent for either one, or both, of a diabetic vascular disorder and a diabetic respiratory disorder. Further alternatively, the present invention relates to the use of a cyclohexenone long chain alcohol compound represented by formula (I) in the prevention and/or the treatment of either one, or both, of a diabetic vascular disorder and a diabetic respiratory disorder. Furthermore, the present invention provides medical packs comprising the following components i and ii:

i. a pharmaceutical composition comprising a cyclohexenone long chain alcohol compound represented by formula (1) and a pharmaceutically acceptable carrier, and

ii. instructions which describe that the above pharmaceutical composition can be used in either one, or both, of the prevention and the treatment of at least one disorder selected from the group consisting of diabetic vascular disorders and diabetic respiratory disorders.

In addition to the subject of treatment, the instructions of the present invention may describe the date of manufacture of the pharmaceutical composition and storage conditions. Furthermore, such information may also be indicated directly on the pharmaceutical composition. Specifically, necessary information can be indicated by directly printing on a container containing the pharmaceutical composition, or by pasting a label thereon. That is, the present invention relates to pharmaceutical compositions comprising a cyclohexenone long chain alcohol compound represented by formula (1) and a pharmaceutically acceptable carrier, with instructions showing that the pharmaceutical composition can be used in either one, or both, of the prevention and treatment of at least one disorder selected from the group consisting of diabetic vascular disorders and diabetic respiratory disorders.

### BRIEF DESCRIPTION OF THE DRAWINGS

FIG. 1 depicts the contractile force (g/mg tissue) of carbachol-treated bronchial smooth muscles of STZ-induced diabetic rat model, showing the mean values±standard deviations (n=6) of the non-treated group, group administered with compound 24 at 8 mg/kg (indicated as “compound 24: 8 mg/kg”), group administered with compound 24 at 2 mg/kg (indicated as “compound 24: 2 mg/kg”), and the control (normal) group. The left graph shows the results for mucosal epithelium (+) samples (TR/E(+)), and the right graph shows the results for mucosal epithelium (−) samples (TR/E(−)).

FIG. 2 depicts the contractile force (g/mg tissue) of vascular U-46619-treated smooth muscles of STZ-induced diabetic rat model, showing the mean values±standard deviations (n=6) of the non-treated group, group administered with compound 24 at 8 mg/kg (indicated as “compound 24: 8 mg/kg”), group administered with compound 24 at 2 mg/kg (indicated as “compound 24: 2 mg/kg”), and the control group. The left graph shows the results for endothelium (+) samples Aor/E(+)), and the right graph shows the results for endothelium (−) samples (Aor/E(−)).

### BEST MODE FOR CARRYING OUT THE INVENTION

In the above formula (1), X represents a linear or branched C10-C28 alkylene or alkenylene group. Examples of the side chains of the branched alkylene or alkenylene group include C1-C10 alkyl groups. The side chain alkyl groups can be selected from the group consisting of a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group, a hexyl group, an isohexyl group, a heptyl group, an octyl group, a nonyl group, a decyl group and the like. Of these side chain alkyl groups, a methyl group is particularly preferred.

In the present invention, the linear alkenylene group includes an alkene structure having at least one carbon-carbon double bond. Moreover, the alkylene group and alkenylene group may include substituents at either position 3 or 7, or both, of the side chains. In other words, if X in formula (1) is a linear alkylene group or a linear alkenylene group, side chains may be included at either one, or both, of positions 3 and 7.

A linear C10-C28 alkylene group is more preferred for X, and a linear C10-C18 alkylene group is particularly preferred in the present invention. Moreover, R¹, R², and R³ each represent a hydrogen atom or a methyl group. It is more preferred that at least any one of the R¹, R², and R³ is a methyl group. Furthermore, compounds in which R¹, R², and R³ are all methyl groups are more preferred compounds in the present invention. In another embodiment, cases in which R¹ and R² are both hydrogen atoms are also preferred. The compound of formula (1) has a variety of possible isomers. These isomers are also encompassed by the present invention.

The method for obtaining a compound represented by formula (1) is publicly known. For example, the compound can be produced according to the process described in JP-A 2000-297034. More specifically, a compound represented by formula (1) can be produced according to, for example, the following process A or B.

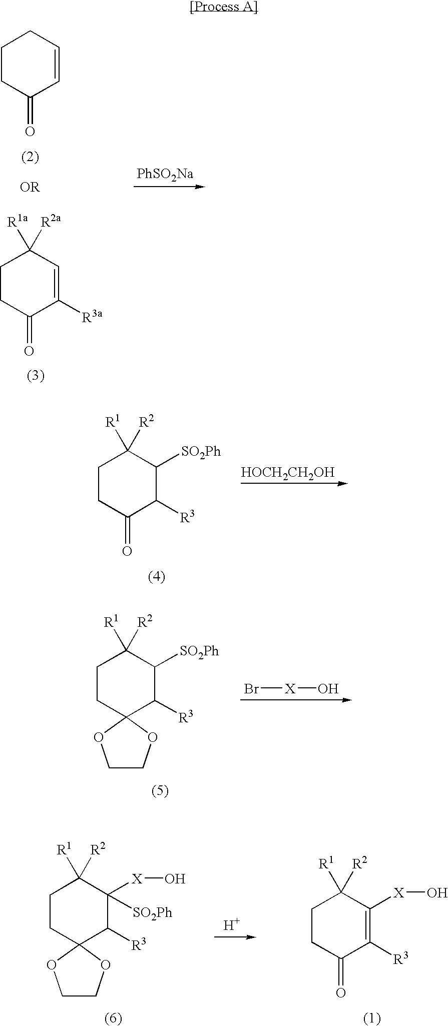

[wherein R^{1a}, R^{2a}, and R^{3a} represent a hydrogen atom or a methyl group. At least one of R^{1a}, R^{2a}, and R^{3a} represents a methyl group. Ph represents a phenyl group, and X, R¹, R², and R³ have the same meanings as defined above.]

That is, cyclohexenone (2) or methyl-substituted-2-cyclohexen-1-one (3) is reacted with a benzenesulfinic acid salt in the presence of an acid, to yield compound (4). The resulting compound (4) is reacted with ethylene glycol to obtain its ketal derivative (5), which is further reacted with ω-halogenoalkanol or ω-halogenoalkenol to yield compound (6). The obtained compound (6) is subjected to an acid treatment to eliminate the protective group, to obtain compound (1).

The methyl-substituted-2-cyclohexen-1-one (3) used here as a raw material can be obtained by reacting methyl-substituted cyclohexanone with a trialkylsilyl halide in the presence of butyl lithium, followed by oxidation in the presence of a palladium-based catalyst.

First, a reaction between cyclohexenone (2) or methyl-substituted-2-cyclohexen-1-one (3) and a benzenesulfinic acid salt, for example, benzenesulfinic acid sodium is preferably performed in the presence of an acid such as hydrochloric acid, sulfuric acid, and phosphoric acid at 0° C. to 100° C. for 5 to 40 hours.

The reaction between compound (4) and ethylene glycol is preferably performed in the presence of a condensing agent such as paratoluenesulfonic anhydride at 50° C. to 120° C. for 1 to 10 hours.

The ω-halogenoalkanol or ω-halogenoalkenol to be reacted with the ketal derivative (5) is preferably ω-bromoalkanol or ω-bromoalkenol. The reaction between the ketal derivative (5) and ω-halogenoalkanol or ω-halogenoalkenol is preferably performed in the presence of a metal compound such as butyl lithium in low-temperature conditions.

The phenylsulfonyl group and the ketal-protective group of the obtained compound (6) can be eliminated by reacting compound (6) with an acid such as paratoluenesulfonic acid.

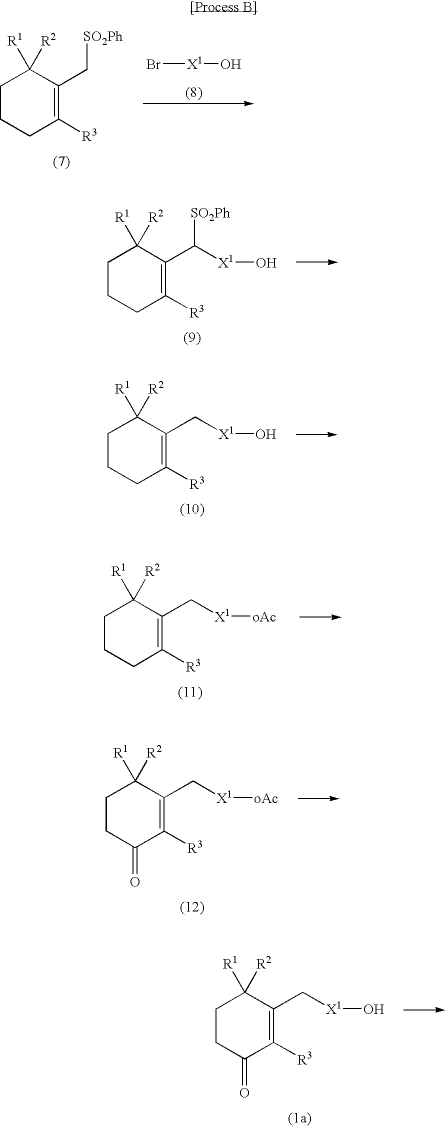

[wherein X¹ represents C9-C27 alkylene group or alkenylene group, Ac represents an acyl group, and R¹, R², R³, and Ph have the same meanings as defined above.]

That is, compound (7) is reacted with ω-bromoalcohol to yield compound (9), followed by elimination of the phenylsulfonyl group to obtain compound (10). Compound (7) can be obtained in accordance with, for example, Synthesis, 1996, Nov. The hydroxy group of the obtained compound (10) is protected to yield compound (11), followed by oxidation to yield compound (12). Furthermore, the hydroxy-protective group of compound (12) is eliminated to thereby obtain compound (1a).

The reaction between compound (7) and compound (8) is preferably performed in the presence of a metal compound such as butyl lithium under low-temperature conditions. The phenylsulfonyl group can be eliminated from compound (9) preferably by reacting compound (9) with, for example, a phosphate salt in the presence of sodium amalgam. The hydroxy-protective group of compound (10) is preferably an acetyl group or the like, and the protection reaction is performed, for example, by reacting compound (10) with acetic anhydride. The oxidation reaction of compound (11) is performed by reacting compound (11) with an alkyl hydroperoxide such as t-butyl hydroperoxide in the presence of a metal compound such as ruthenium trichloride. The protective group can be eliminated from compound (12) preferably by hydrolyzing compound (12) in the presence of a base such as potassium carbonate.

The present invention provides preventive agents for either one, or both, of a diabetic vascular disorder and a diabetic respiratory disorder comprising, as an active ingredient, a cyclohexenone long chain alcohol compound represented by formula (1). Moreover, the present invention relates to methods for preventing either one, or both, of a diabetic vascular disorder and a diabetic respiratory disorder comprising the step of administering a cyclohexenone long chain alcohol compound represented by formula (1).

Furthermore, the present invention provides therapeutic agents for either one, or both, of a diabetic vascular disorder and a diabetic respiratory disorder comprising, as an active ingredient, a cyclohexenone long chain alcohol compound represented by formula (1). Alternatively, the present invention relates to methods for treating either one, or both, of a diabetic vascular disorder and a diabetic respiratory disorder comprising the step of administering a cyclohexenone long chain alcohol compound represented by formula (1).

In the present invention, the terms “treatment” and “therapy” include prevention. In patients with chronic diseases such as diabetes, the pathological condition is continuous. Therefore, a drug administered for the purpose of treatment gives a therapeutic effect on the pathological condition that has been continuing from prior to administration. At the same time, the drug preventively acts on pathological condition(s) that occur after administration. That is, the present invention provides a preventive and therapeutic agent for either one, or both, of a diabetic vascular disorder and a diabetic respiratory disorder comprising, as an active ingredient, a cyclohexenone long chain alcohol compound represented by formula (1). Alternatively, the present invention relates to a method for preventing and treating either one, or both, of a diabetic vascular disorder and a diabetic respiratory disorder comprising the step of administering a cyclohexenone long chain alcohol compound represented by formula (1).

Alternatively, the present invention relates to the use of a cyclohexenone long chain alcohol compound represented by formula (1) in the production of pharmaceutical compositions for treating either one, or both, of a diabetic vascular disorder and a diabetic respiratory disorder. Furthermore, the present invention relates to the use of a cyclohexenone long chain alcohol compound represented by formula (1) in the treatment of either one, or both, of a diabetic vascular disorder and a diabetic respiratory disorder.

Moreover, the present invention relates to the use of a cyclohexenone long chain alcohol compound represented by formula (1) in the production of pharmaceutical compositions for preventing either one, or both, of a diabetic vascular disorder and a diabetic respiratory disorder. Furthermore, the present invention relates to the use of a cyclohexenone long chain alcohol compound represented by formula (1) in the prevention of either one, or both, of a diabetic vascular disorder and a diabetic respiratory disorder. Alternatively, the present invention relates to the use of a cyclohexenone long chain alcohol compound represented by formula (1) in the production of pharmaceutical compositions for preventing and treating either one, or both, of a diabetic vascular disorder and a diabetic respiratory disorder. Furthermore, the present invention relates to the use of a cyclohexenone long chain alcohol compound represented by formula (1) in the prevention and treatment of either one, or both, of a diabetic vascular disorder and a diabetic respiratory disorder.

Diabetic vascular disorders are typically classified into microangiopathies or macroangiopathies. The term “diabetic vascular disorders” in the present invention includes microangiopathies and macroangiopathies. Diabetic complications brought on by these angiopathies include the following diseases. Therefore, the present invention is useful for preventing and/or treating the following diabetic complications.

Complications accompanying blood flow disorders
Hemorheological changes and accumulation of Maillard reaction products

### Opthalmopathies

diabetic retinopathy and ischemic optic neuropathy
   Cardiovascular disorders
ischemic heart diseases (myocardial infarction, coronary arteriosclerosis, and angina pectoris) and diabetic cardiomyopathy
   Cerebrovascular disorders
cerebral infarction and dementia
   Skin lesions caused by peripheral vascular disorders
pretibial pigmented patches, diabetic blisters, disseminated granuloma annulare, and necrobiosis lipoidica
   Diabetic gangrenes/ulcers and skin lesions caused by arteriosclerosis
   Diabetic osteopathies caused by ischemia

For example, microangiopathies are pathological conditions involving microvasculature (capillary vessel) lesions. Diabetic retinopathy, which is a typical diabetic complication, is a pathological condition brought on by microangiopathy. Thus, the present invention is useful for preventing and treating diabetic retinopathy. Meanwhile, macroangiopathies are arteriosclerotic vascular disorders. Foot lesions such as foot ulcers and gangrenes, which, similar to retinopathy, are typical diabetic complications that are closely related to macroangiopathies. Therefore, the present invention is useful for preventing and treating foot lesions associated with diabetes.

Further, the term “diabetic respiratory disorders” of the present invention includes sleep apnea syndrome in diabetic patients. It is known that diabetic patients often concurrently develop the sleep apnea syndrome (herein below abbreviated as SAS). In fact, patients diagnosed with SAS are frequently found to be diabetic patients. For example, it has been reported that juvenile diabetic patients often have SAS (Ann. Neurol. 17, 391-395, 1985). Obesity, high blood pressure or the like, as well as organic causes such as the shape of the airway have been pointed out to be associated with SAS. Airway narrowing caused by a diabetic autonomic disorder is considered to be one of the causatives of SAS in diabetic patients.

At present, nasal continuous positive airway pressure (nasal CPAP) treatment is known to be the most effective therapeutic method for SAS. Nasal CPAP treatment is a therapeutic method for preventing the occurrence of apnea by maintaining the upper airway at positive pressure at all times with a special device. Nasal CPAP treatment provides a great therapeutic effect; however it requires patients to wear the device while they sleep. Thus, it would be useful if SAS treatment by drug administration is realized.

Cyclohexenone long chain alcohol compounds represented by formula (1) act on airway smooth muscle to inhibit its contraction. Therefore, diabetic respiratory disorders such as SAS can be prevented and treated by administrating the compounds. In other words, the present invention includes preventive and/or therapeutic agents for diabetic SAS comprising, as an active ingredient, a cyclohexenone long chain alcohol compound represented by formula (1).

Furthermore, it has been implied that diabetic SAS may contribute to insulin resistance. Thus, a therapeutic effect on insulin resistance can be expected by improving SAS. In fact, it has been shown that sugar tolerance decreases with the aggravation of SAS. Moreover, there is also a report that insulin resistance was improved by treating SAS with nasal CPAP treatment (J. Clin. Endocrinol. Med. 79/6, 1681-1685, 1994). Therefore, an improving effect on insulin resistance can be expected by treating a diabetic respiratory disorder according to the present invention.

That is, the present invention provides methods for treating diabetic sleep apnea syndrome, comprising the step of administering a cyclohexenone long chain alcohol compound having the structure of formula (1), to a patient with diabetic sleep apnea syndrome.

Not only stimuli of neurotransmitters released from nerve endings, but also mechanisms such as signal transduction from muscles to contractile proteins and calcium inflow into cells are involved in smooth muscle contraction (edited by Kazunari Takayanagi, Manual of Pharmacology, published by Nanzando, pp. 23, 1989). Sakai Y et. al. have reported that abnormal contraction in diabetic rats was associated with calcium behavior in smooth muscle and phosphatidylinositol turnover (Sakai Y et. al., Eur. J. Pharmacol., 162/3, 475-481, 1989). Compound (1) of the present invention may act on these mechanisms to inhibit abnormal contraction of smooth muscle, thereby preventing blood vessel and airway narrowing.

Compound (1) may be administered by either an oral administration or a parenteral administration (such as intramuscular administration, subcutaneous administration, intravenous administration, and administration by a suppository).

If a formulation for oral administration is to be prepared, excipients and other additives such as binders, disintegrators, lubricants, colorants, and flavoring agents are added as required, and then formed into tablets, coated-tablets, granules, capsules, solutions, syrups, elixirs, oily or aqueous suspensions, or the like by ordinary methods. Examples of excipients include lactose, corn starch, saccharose, glucose, sorbitol, and crystalline cellulose. Examples of binders include polyvinyl alcohol, polyvinyl ether, ethyl cellulose, methyl cellulose, gum arabic, tragacanth, gelatin, shellac, hydroxypropyl cellulose, hydroxypropyl starch, and polyvinyl pyrrolidone.

Examples of disintegrators include starch, agar, gelatin powder, crystalline cellulose, calcium carbonate, sodium bicarbonate, calcium citrate, dextran, and pectin. Examples of lubricants include magnesium stearate, talc, polyethylene glycol, silica, and hardened vegetable oil. Colorants that are permitted to be added to pharmaceuticals may be used. Examples of flavoring agents which may be used include cocoa powder, menthol crystals, aromatic acids, menthol oil, borneol, cinnamon powder, menthol, peppermint oil, and camphor. These tablets or granules may be appropriately coated with sugar, gelatin, and other coatings as required.

If an injection is prepared, pH-regulators, buffers, stabilizers, preservatives, and the like are added as required, and formed into a formulation for subcutaneous, intramuscular, or intravenous injection, by ordinary methods. The injection may be prepared as needed as a solid formulation by, for example, lyophilizing the solution after it is stored in a container. Moreover, a single dose may be stored in a container, or several doses may be stored in the same container.

When the compound of the present invention is administered as a pharmaceutical, the daily dose for a human adult is typically within the range of 0.01 to 1000 mg, and preferably, 0.1 to 500 mg. The daily dose is administered either at a single time or in 2 to 4 divided doses.

Herein below, the present invention will further be described with reference to Examples.

All prior art documents cited in the present specification are incorporated herein by reference.

### EXAMPLES

### Preparation Example 1

(1) 10.25 g of benzenesulfinic acid sodium was added to a solution having 5 ml of cyclohexenone and 30 ml of water. To this solution, 60 ml of 1 N hydrochloric acid was added dropwise. After stirring at room temperature for 24 hours, the crystals thus deposited were filtered, and washed with water, isopropanol, and cold ether. After recrystallization with isopropanol, 5.74 g of 3-(phenylsulfonyl)-cyclohexan-1-one was obtained in the form of white crystals (Melting Point, 83° C. to 85° C.; Yield, 97%).

(2) 0.3 ml of 1,2-ethanediol and 0.2 g of anhydrous paratoluenesulfonic acid were added to a solution having 5.3 g of 3-(phenylsulfonyl)-cyclohexan-1-one dissolved in 60 ml of benzene. The reaction solution was heated under reflux for four hours. After the reaction, 2 M aqueous sodium bicarbonate solution was added thereto, and extraction was done three times with ethyl acetate. The organic phase was washed with saturated saline, and then dried over magnesium sulfate. The solvent was distilled off under reduced pressure. Then, the residue was recrystallized with ether, to thereby obtain 6.1 g of 1,1-(ethylenedioxy)-3-(phenylsulfonyl)-cyclohexane in the form of white crystals (Melting Point, 93° C. to 95° C.; Yield, 97%).

(3) 2 ml of n-butyl lithium solution was added dropwise to 5 ml of THF (tetrahydro furan) solution having 565 mg of 1,1-(ethylenedioxy)-3-(phenylsulfonyl)-cyclohexane and 4 mg of triphenylmethane at −78° C. under an argon stream. After stirring for 10 minutes, the reaction was effected at room temperature for one hour. 1 ml of HMPT (hexamethyl phosphoric triamide) was added thereto. The resulting solution was recooled to −78° C., and 2 ml of THF solution having 159 mg of 10-bromo-1-decanol was added dropwise. After reaction at −20° C. for two hours, the reaction solution was poured into a saturated ammonium chloride solution. The solution was extracted with ether. The organic phase was washed with water and saturated saline, and then dried over magnesium sulfate. The solvent was distilled off under reduced pressure. Then, the residue was purified by silica gel column chromatography using hexane-ethyl acetate (AcOEt), to thereby obtain 265 mg of 1,1-(ethylenedioxy)-3-(10-hydroxydecyl)-3-(phenylsulfonyl)-cyclohexane in the form of a colorless oil (Yield, 90%).

(4) 20 mg of paratoluenesulfonic acid was added to a solution having 193 mg of 1,1-(ethylenedioxy)-3-(10-hydroxydecyl)-3-(phenylsulfonyl)-cyclohexane in 3 ml of chloroform and 0.6 ml of acetone. The mixed solution was reacted at 50° C. for 24 hours. 10 ml of saturated aqueous sodium bicarbonate solution was added thereto, and subjected to dichloromethane extraction. The organic phase was washed with saturated saline, and then dried over magnesium sulfate. The solvent was distilled off under reduced pressure. Then, the residue was purified by silica gel column chromatography using hexane-ethyl acetate, to thereby obtain 86 mg of 3-(10-hydroxydecyl)-2-cyclohexen-1-one (3-(10-hydroxydecyl)-2-cyclohexenone) in the form of a colorless oil (Yield, 77%).

In a manner similar to Preparation Example 1, the following compounds were obtained.

### Preparation Example 2

3-(11-hydroxyundecyl)-2-cyclohexen-1-one (3-(11-hydroxyundecyl)-2-cyclohexenone) (Melting Point, 34° C. to 35° C.).

### Preparation Example 3

3-(12-hydroxydodecyl)-2-cyclohexen-1-one (3-(12-hydroxydodecyl)-2-cyclohexenone) (Melting Point, 35° C. to 36° C.)

### Preparation Example 4

3-(13-hydroxytridecyl)-2-cyclohexen-1-one (3-(13-hydroxytridecyl)-2-cyclohexenone) (Melting Point, 42° C. to 43° C.)

### Preparation Example 5

3-(14-hydroxytetradecyl)-2-cyclohexen-1-one (3-(14-hydroxytetradecyl)-2-cyclohexenone) (Melting Point, 44° C. to 45° C.)

### Preparation Example 6

(1) 35.4 ml of 1.4 M n-butyl lithium solution was added dropwise to 20 ml of THF solution having 7 ml of N,N-diisopropylamine at −78° C. The solution was stirred at 0° C. for 30 minutes. This LDA (lithium diisopropylamide) solution was added dropwise to 10 ml of THF solution having 4 ml of 4-methylcyclohexane-1-one at −78° C. After stirring at −78° C. for one hour, 6.5 ml of trimethylsilyl chloride was added thereto. After stirring at room temperature for one hour, the solution was poured into an aqueous sodium bicarbonate solution, and extracted with ether. The organic phase was washed with saturated saline, and then dried over magnesium sulfate. The solvent was distilled off under reduced pressure. Then, the residue was purified by vacuum distillation, to thereby obtain 5.83 g of 4-methyl-1-(trimethylsilyloxy)-1-cyclohexene (thin layer chromatography/TLC:(hexane-AcOEt:8-2) Rf=0.8; Yield, 96%).

(2) A catalyst amount of palladium acetate was added to 70 ml of DMSO (dimethylsulfoxide) solution having 3.53 g of 4-methyl-1-(trimethylsilyloxy)-1-cyclohexene, followed by stirring while introducing oxygen for six hours. Water was added at 0° C., and the solution was filtered over celite, and then extracted with ether. The solvent of the organic phase was distilled off under reduced pressure and the residue was dissolved in hexane-water, and the resulting solution was extracted with hexane. The hexane phase was washed with saturated saline and dried over magnesium sulfate. The solvent was distilled off under reduced pressure, to thereby obtain 4-methyl-2-cyclohexen-1-one in the form of an oil (TLC:(hexane-AcOEt:8-2)Rf=0.35; Yield, 72%).

(3) 3.0 g of benzenesulfinic acid sodium was added to a solution containing 1.52 g of 4-methyl-2-cyclohexen-1-one and 9 ml of water. 18 ml of 1 N hydrochloric acid was added dropwise to this solution. After stirring at room temperature for 24 hours, the crystals thus deposited were filtered, and washed with water, isopropanol, and cold ether. After recrystallization with isopropanol, 4-methyl-3-(phenylsulfonyl)-cyclohexan-1-one (Melting Point, 71° C. to 74° C.) was obtained in the form of white crystals (Yield, 72%).

(4) 0.7 ml of 1,2-ethanediol and 0.2 g of anhydrous paratoluenesulfonic acid were added to a solution having 2.45 g of 4-methyl-3-(phenylsulfonyl)-cyclohexan-1-one in 40 ml of benzene. The reaction solution was heated under reflux for four hours. After the reaction, 2 M aqueous sodium bicarbonate solution was added thereto, and extraction done three times with ethyl acetate. The organic phase was washed with saturated saline, and then dried over magnesium sulfate. The solvent was distilled off under reduced pressure. Then, the residue was recrystallized with ether, to thereby obtain 1,1-(ethylenedioxy)-4-methyl-3-(phenylsulfonyl)-cyclohexane (Melting Point, 105° C. to 106° C.) in the form of white crystals (Yield, 97%).

(5) 1.8 ml solution of n-butyl lithium was added dropwise to 5 ml of THF solution having 560 mg of 1,1-(ethylenedioxy)-4-methyl-3-(phenylsulfonyl)-cyclohexane and 4 mg of triphenylmethane at −78° C. under an argon stream. After stirring for 10 minutes, the reaction was effected at room temperature for one hour. 1 ml of HMPT was added thereto. The resulting solution was recooled to −78° C., and 2 ml of THF solution having 166 mg of 10-bromo-1-decanol was added dropwise. After reaction at −20° C. for two hours, the reaction solution was poured into a saturated ammonium chloride solution. The solution was extracted with ether. The organic phase was washed with water and saturated saline, and then dried over magnesium sulfate. The solvent was distilled off under reduced pressure. The residue was then purified by silica gel column chromatography using hexane-ethyl acetate, to thereby obtain 1,1-(ethylenedioxy)-3-(10-hydroxydecyl)-4-methyl-3-(phenylsulfonyl)-cyclohexane in the form of a colorless oil (TLC:(hexane-AcOEt:6-4)Rf=0.14; Yield, 97%).

(6) 20 mg of paratoluenesulfonic acid was added to a solution having 235 mg of 1,1-(ethylenedioxy)-3-(10-hydroxydecyl)-4-methyl-3-(phenylsulfonyl)-cyclohexane in 20 ml of chloroform and 4 ml of acetone. The mixed solution was reacted at 50° C. for 24 hours. 10 ml of saturated aqueous sodium bicarbonate solution was added thereto, and extraction done with dichloromethane. The organic phase was washed with saturated saline, and then dried over magnesium sulfate. The solvent was distilled off under reduced pressure. Then, the residue was purified by silica gel column chromatography using hexane-ethyl acetate, to thereby obtain 3-(10-hydroxydecyl)-4-methyl-2-cyclohexen-1-one (3-(10-hydroxydecyl)-4-methyl-2-cyclohexenone) in the form of a colorless oil (TLC:(hexane-AcOEt:6-4)Rf=0.2; Yield, 75%).

In a manner similar to Preparation Example 6, the following compounds were obtained.

### Preparation Example 7

3-(11-hydroxyundecyl)-4-methyl-2-cyclohexen-1-one (3-(11-hydroxyundecyl)-4-methyl-2-cyclohexenone) (TLC:(hexane-AcOEt:6-4)Rf=0.21)

### Preparation Example 8

3-(12-hydroxydodecyl)-4-methyl-2-cyclohexen-1-one (3-(12-hydroxydodecyl)-4-methyl-2-cyclohexenone) (TLC:(hexane-AcOEt:6-4)Rf=0.22)

### Preparation Example 9

3-(13-hydroxytridecyl)-4-methyl-2-cyclohexen-1-one (3-(13-hydroxytridecyl)-4-methyl-2-cyclohexenone) (TLC:(hexane-AcOEt:6-4)Rf=0.25)

### Preparation Example 10

3-(14-hydroxytetradecyl)-4-methyl-2-cyclohexen-1-one (3-(14-hydroxytetradecyl)-4-methyl-2-cyclohexenone) (TLC:(hexane-AcOEt:6-4)Rf=0.3)

### Preparation Example 11

(1) 5.98 g of benzenesulfinic acid sodium was added to a solution having 3 ml of 4,4-dimethyl-2-cyclohexen-1-one and 30 ml of water. 40 ml of 1 N hydrochloric acid was added dropwise to this solution. After stirring at room temperature for 24 hours, the crystals thus deposited were filtered, and washed with water, isopropanol, and cold ether. After recrystallization with isopropanol, 4,4-dimethyl-3-(phenylsulfonyl)-cyclohexan-1-one was obtained in the form of white crystals (Melting Point, 84° C. to 86° C.; Yield, 89%).

(2) 1.1 ml of 1,2-ethanediol and 0.3 g of anhydrous paratoluenesulfonic acid were added to a solution having 4.4 g of 4,4-dimethyl-3-(phenylsulfonyl)-cyclohexan-1-one dissolved in 45 ml of benzene. The reaction solution was heated under reflux for four hours. After the reaction, 2 M aqueous sodium bicarbonate solution was added thereto, and extracted with ethyl acetate three times. The organic phase was washed with saturated saline, and then dried over magnesium sulfate. The solvent was distilled off under reduced pressure. Then, the residue was recrystallized with ether, to thereby obtain 4,4-dimethyl-1,1-(ethylenedioxy)-3-(phenylsulfonyl)-cyclohexane in the form of white crystals (Melting Point, 113° C. to 115° C.; Yield, 84%).

(3) 2.93 ml of n-butyl lithium solution was added dropwise to 5 ml of THF solution having 930 mg of 4,4-dimethyl-1,1-(ethylenedioxy)-3-(phenylsulfonyl)-cyclohexane and 4 mg of triphenylmethane at −78° C. under an argon stream. After stirring for 10 minutes, the reaction was effected at room temperature for one hour. 1 ml of HMPT was added thereto. The resulting solution was recooled to −78° C., and 2 ml of THF solution having 236 mg of 10-bromo-1-decanol was added dropwise. After reaction at −20° C. for two hours, the reaction solution was poured into a saturated ammonium chloride solution, and extracted with ether. The organic phase was washed with water and saturated saline, and then dried over magnesium sulfate. The solvent was distilled off under reduced pressure. Then, the residue was purified by silica gel column chromatography using hexane-ethyl acetate, to thereby obtain 4,4-dimethyl-1,1-(ethylenedioxy)-3-(10-hydroxydecyl)-3-(phenylsulfonyl)-cyclohexane in the form of a colorless oil (TLC:(hexane-AcOEt:6-4)Rf=0.15; Yield, 94%).

(4) 20 mg of paratoluenesulfonic acid was added to a solution having 400 mg of 4,4-dimethyl-1,1-(ethylenedioxy)-3-(10-hydroxydecyl)-3-(phenylsulfonyl)-cyclohexane in 30 ml of chloroform and 6 ml of acetone. The mixed solution was reacted at 50° C. for 24 hours. 10 ml of saturated aqueous sodium bicarbonate solution was added thereto, and extracted with dichloromethane. The organic phase was washed with saturated saline, and then dried over magnesium sulfate. The solvent was distilled off under reduced pressure. Then, the residue was purified by silica gel column chromatography using hexane-ethyl acetate, to thereby obtain 4,4-dimethyl-3-(10-hydroxydecyl)-2-cyclohexen-1-one (4,4-dimethyl-3-(10-hydroxydecyl)-2-cyclohexenone) in the form of a colorless oil (TLC:(hexane-AcOEt:6-4)Rf=0.25; Yield, 78%).

In a manner similar to Preparation Example 11, the following compounds were obtained.

### Preparation Example 12

3-(11-hydroxyundecyl)-4,4-dimethyl-2-cyclohexen-1-one (3-(11-hydroxyundecyl)-4,4-dimethyl-2-cyclohexenone) (TLC:(hexane-AcOEt:6-4)Rf=0.25)

### Preparation Example 13

3-(12-hydroxydodecyl)-4,4-dimethyl-2-cyclohexen-1-one (3-(12-hydroxydodecyl)-4,4-dimethyl-2-cyclohexenone) (TLC:(hexane-AcOEt:6-4)Rf=0.27)

### Preparation Example 14

3-(13-hydroxytridecyl)-4,4-dimethyl-2-cyclohexen-1-one (3-(13-hydroxytridecyl)-4,4-dimethyl-2-cyclohexenone) (TLC:(hexane-AcOEt:6-4)Rf=0.3)

### Preparation Example 15

3-(14-hydroxytetradecyl)-4,4-dimethyl-2-cyclohexen-1-one (3-(14-hydroxytetradecyl)-4,4-dimethyl-2-cyclohexenone) (TLC:(hexane-AcOEt:6-4)Rf=0.3)

### Preparation Example 16

(1) 2.9 g of benzenesulfinic acid sodium was added to a solution having 1.5 g of 2-methyl-2-cyclohexen-1-one and 8 ml of water. 16 ml of 1 N hydrochloric acid was added dropwise to this solution. After stirring at room temperature for 24 hours, the crystals thus deposited were filtered, and washed with water, isopropanol, and cold ether. After recrystallization with isopropanol, 2-methyl-3-(phenylsulfonyl)-cyclohexan-1-one was obtained in the form of white crystals (TLC:(hexane-AcOEt:6-4)Rf=0.25; Yield, 93%).

(2) 0.41 ml of 1,2-ethanediol and 0.1 g of anhydrous paratoluenesulfonic acid were added to a solution having 1.4 g of 2-methyl-3-(phenylsulfonyl)-cyclohexan-1-one dissolved in 20 ml of benzene. The reaction solution was heated under reflux for four hours. After the reaction, 2 M aqueous sodium bicarbonate solution was added thereto, and subjected to ethyl acetate extraction three times. The organic phase was washed with saturated saline, and then dried over magnesium sulfate. The solvent was distilled off under reduced pressure. Then, the residue was recrystallized with ether, to thereby obtain 1,1-(ethylenedioxy)-2-methyl-3-(phenylsulfonyl)-cyclohexane in the form of white crystals (Melting Point, 76° C. to 77° C.; Yield, 95%).

(3) 1.02 ml of n-butyl lithium solution was added dropwise to 5 ml of THF solution having 304 mg of 1,1-(ethylenedioxy)-2-methyl-3-(phenylsulfonyl)-cyclohexane and 4 mg of triphenylmethane at −78° C. under an argon stream. After stirring for 10 minutes, the reaction was effected at room temperature for one hour. 1 ml of HMPT was added thereto. The resulting solution was recooled to −78° C., and 2 ml of THF solution having 90 mg of 10-bromo-1-decanol was added dropwise. After the reaction at −20° C. for two hours, the reaction solution was poured into a saturated ammonium chloride solution. The solution was extracted with ether. The organic phase was washed with water and saturated saline, and then dried over magnesium sulfate. The solvent was distilled off under reduced pressure. Then, the residue was purified by silica gel column chromatography using hexane-ethyl acetate, to thereby obtain 1,1-(ethylenedioxy)-3-(10-hydroxydecyl)-2-methyl-3-(phenylsulfonyl)-cyclohexane in the form of a colorless oil (TLC:(hexane-AcOEt:6-4)Rf=0.2; Yield, 92%).

(4) 20 mg of paratoluenesulfonic acid was added to a solution having 388 mg of 1,1-(ethylenedioxy)-3-(10-hydroxydecyl)-2-methyl-3-(phenylsulfonyl)-cyclohexane in 30 ml of chloroform and 6 ml of acetone. The mixed solution was effected a reaction at 50° C. for 24 hours. 10 ml of saturated aqueous sodium bicarbonate solution was added thereto, and subjected to a dichloromethane extraction. The organic phase was washed with saturated saline, and then dried over magnesium sulfate. The solvent was distilled off under reduced pressure. Then, the residue was purified by silica gel column chromatography using hexane-ethyl acetate, to thereby obtain 3-(10-hydroxydecyl)-2-methyl-2-cyclohexen-1-one (3-(10-hydroxydecyl)-2-methyl-2-cyclohexenone) in the form of a colorless oil (TLC:(hexane-AcOEt:6-4)Rf=0.2; Yield, 45%).

In a manner similar to Preparation Example 16, the following compounds were obtained.

### Preparation Example 17

3-(11-hydroxyundecyl)-2-methyl-2-cyclohexen-1-one
(3-(11-hydroxyundecyl)-2-methyl-2-cyclohexenone) (TLC:(hexane-AcOEt:6-4)Rf=0.24)

### Preparation Example 18

3-(12-hydroxydodecyl)-2-methyl-2-cyclohexen-1-one
(3-(12-hydroxydodecyl)-2-methyl-2-cyclohexenone) (TLC: (hexane-AcOEt:6-4) Rf=0.26)

### Preparation Example 19

3-(13-hydroxytridecyl)-2-methyl-2-cyclohexen-1-one
(3-(13-hydroxytridecyl)-2-methyl-2-cyclohexenone) (TLC:(hexane-AcOEt:6-4)Rf=0.28)

### Preparation Example 20

3-(14-hydroxytetradecyl)-2-methyl-2-cyclohexen-1-one
(3-(14-hydroxytetradecyl)-2-methyl-2-cyclohexenone) (TLC: (hexane-AcOEt:6-4)Rf=0.3)

### Preparation Example 21

(1) 4 ml of hexane solution with n-butyl lithium (1.4 M) was added to dry THF solution (8 ml) containing 1 g of 1-phenylsulfonylmethyl-2,6,6-trimethyl-1-cyclohexene and 4 mg of triphenylmethane at −78° C. under an argon gas atmosphere. After stirring for 10 minutes, 1.5 ml of hexamethylphosphoric triamide was added under stirring at room temperature. After 1.5 hours at this temperature, the mixture was cooled to −78° C. and 439 mg of 11-bromoundecanol was added slowly. The mixture was stirred for three hours at −20° C. and poured into 40 ml of saturated ammonium chloride solution. The obtained solution was extracted with ether. The organic phase was washed with saline, and then dried over magnesium sulfate. The solvent was distilled off under reduced pressure. The residue was purified by silica gel column chromatography, to thereby obtain 622 mg of 1-(12-hydroxydodecyl-1-phenylsulfonyl)-2,6,6-trimethyl-1-cyclohexene as a white solid (TLC:(hexane-AcOEt:6-4)Rf=0.43).

(2) 366 mg of Na₂HPO₄ and 4 g of mercury-sodium amalgam were added to 25 ml of dry ethanol solution containing 579 mg of 1-(12-hydroxydodecyl-1-phenylsulfonyl)-2,6,6-trimethyl-1-cyclohexene at 0° C. under an argon gas atmosphere. The mixture was stirred at room temperature for one hour, then cooled with 5% HCl, and extracted with ether. The organic phase was washed with water, and then dried over magnesium sulfate. The solvent was distilled off under reduced pressure. The hydroxyl group of the residue was acetylated according to the usual method, to thereby obtain 353 mg of 1-(12-acetoxydodecyl)-2,6,6-trimethyl-1-cyclohexene as a colorless oil (TLC:(hexane-AcOEt:5-5)Rf=0.75).

(3) 0.8 ml of water, 1.3 mg of ruthenium trichloride hydrate, and 1.26 ml of 70% t-BuOOH was added to 6 ml of cyclohexane solution containing 321 mg of 1-(12-acetoxydodecyl)-2,6,6-trimethyl-1-cyclohexene. The solution was stirred at room temperature for six hours, and was filtered through celite. The filtrate was added to a 10% Na₂SO₃ solution. The solution was extracted with ether, and the organic phase was washed with saline, and dried over magnesium sulfate. Then, the solvent was distilled off under reduced pressure. The residue was purified by silica gel column chromatography, to thereby obtain 227 mg of 3-(12-acetoxydodecyl)-2,4,4-trimethyl-2-cyclohexen-1-one as a colorless oil (TLC:(hexane-AcOEt:3-7)Rf=0.68).

(4) To a dry methanol solution (8 ml) containing 132 mg of 3-(12-acetoxydodecyl)-2,4,4-trimethyl-2-cyclohexen-1-one, 3 drops of water and 74 mg of K₂CO₃ was added. After stirring at room temperature for 2.5 hours, pH of the solution was adjusted to pH 7 with 5% HCl. The mixture was extracted with ether, and the organic phase was dried over magnesium sulfate. The solvent was distilled off under reduced pressure. The residue was purified by silica gel column chromatography, to thereby obtain 94 mg of 3-(12-hydroxydodecyl)-2,4,4-trimethyl-2-cyclohexen-1-one (3-(12-hydroxydodecyl)-2,4,4-trimethyl-2-cyclohexenone) as a colorless oil (TLC:(hexane-AcOEt:7-3)Rf=0.2).

In a manner similar to Preparation Example 21, the following compounds were obtained.

### Preparation Example 22

3-(13-hydroxytridecyl)-2,4,4-trimethyl-2-cyclohexen-1-one
(3-(13-hydroxytridecyl)-2,4,4-trimethyl-2-cyclohexenone) (TLC:(hexane-AcOEt:7-3)Rf=0.2)

### Preparation Example 23

3-(14-hydroxytetradecyl)-2,4,4-trimethyl-2-cyclohexen-1-one
(3-(14-hydroxytetradecyl)-2,4,4-trimethyl-2-cyclohexenone) (TLC:(hexane-AcOEt:7-3)Rf=0.25)

### Preparation Example 24

3-(15-hydroxypentadecyl)-2,4,4-trimethyl-2-cyclohexen-1-one
(3-(15-hydroxypentadecyl)-2,4,4-trimethyl-2-cyclohexenone)
   (TLC:(hexane-AcOEt:7-3)Rf=0.29)

### Preparation Example 25

3-(16-hydroxyhexadecyl)-2,4,4-trimethyl-2-cyclohexen-1-one
(3-(16-hydroxyhexadecyl)-2,4,4-trimethyl-2-cyclohexenone)
   (TLC:(hexane-AcOEt:7-3)Rf=0.26).

### Example 1

### Nerve Injury-Preventive Effect on Bronchial Smooth Muscle of Streptozotocin-Induced Diabetic Rats

### [Test Method]

Eight-week-old male SD rats were divided into four groups, three of which were intraperitoneally administered with 50 mg/kg of streptozotocin (hereafter, also referred to as STZ) to induce diabetes. Of this diabetic model, two groups were used as the test substance-administered groups, and were intraperitoneally administered with 2 mg/kg or 8 mg/kg of the compound, 3-(15-hydroxypentadecyl)-2,4,4-trimethyl-2-cyclohexen-1-one (hereunder, referred to as “compound 24”) synthesized in Preparation Example 24, once daily for four weeks continuously. The diabetic model which was not administered with “compound 24” was used as the control group, and rats administered with neither STZ nor “compound 24” were used as the non-treated group.

After four weeks from the start of administration, tracheae were isolated to prepare specimens (mucosal epithelium (+) specimens). The mucosal epithelia were further removed from these specimens to form mucosal epithelium (−) specimens. These specimens were suspended in an organ bath, and the contractile response to cumulative administration of carbachol (CAS Registry No.: 51-83-2) was observed. The muscle contractile force was measured with various cumulative concentrations of carbachol within the range of 10^{-8.0} to 10^{-4.5} M, to obtain the maximum muscle contractile force (Emax (g/mg tissue)) and the effective dose for inducing 50% muscle contraction (ED₅₀ (M)).

### [Results]

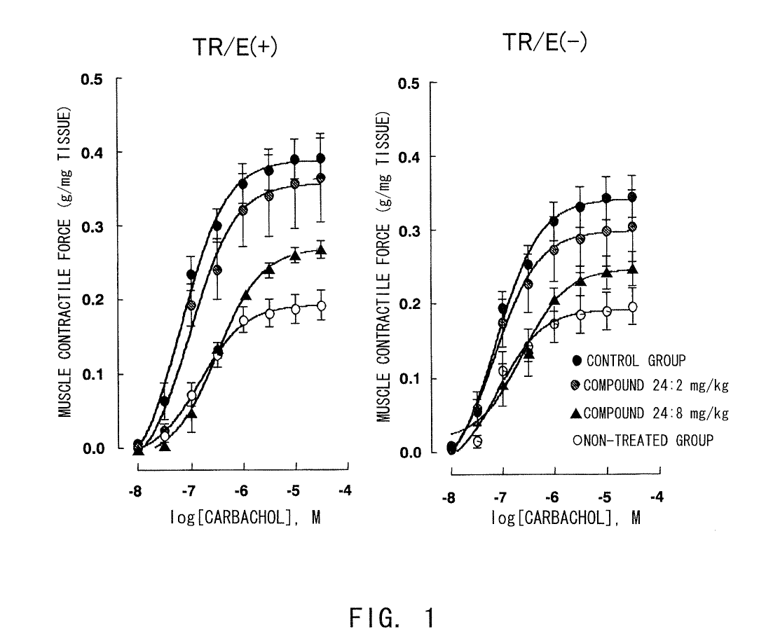

The results are shown in FIG. 1, Table 1, and Table 2. In both mucosal epithelium (+) specimens and mucosal epithelium (−) specimens, the control group showed greater Emax increase than the non-treated group after tracheal smooth muscle contraction was induced with carbachol; however, the administration of “compound 24” tended to suppress the Emax increase concentration-dependently. Moreover, the control group showed greater ED₅₀ decrease than the non-treated group. However, the administration of “compound 24” inhibited the ED₅₀ decrease in a dose-dependent manner. These results showed the tracheal smooth muscle contraction-improving tendency of “compound 24”.

TABLE 1

| | Emax | ED₅₀ |
|---|---|---|
| | (g/mg tissue) | (×10⁻⁸ M) |
| Non-treated group | 0.202 ± 0.017 | 16.6 ± 3.8 |
| Control group | 0.392 ± 0.028 | 11.4 ± 2.3 |
| Compound 24: 2 mg/kg | 0.303 ± 0.047 | 12.2 ± 2.3 |
| Compound 24: 8 mg/kg | 0.292 ± 0.029 | 17.2 ± 5.3 |

Table 1 shows the maximum muscle contractile force (Emax (g/mg tissue)) and the effective dose for inducing 50% muscle contraction (ED₅₀ (M)) of carbachol-treated mucosal epithelium (+) specimens of tracheal smooth muscles from the STZ-induced diabetic rat model. Data is presented as mean values±standard deviations (n=6).

TABLE 2

| | Emax | ED₅₀ |
|---|---|---|
| | (g/mg tissue) | (×10⁻⁸ M) |
| Non-treated group | 0.187 ± 0.016 | 12.7 ± 3.6 |
| Control group | 0.357 ± 0.028 | 9.9 ± 2.4 |
| Compound 24: 2 mg/kg | 0.307 ± 0.036 | 11.4 ± 3.1 |
| Compound 24: 8 mg/kg | 0.242 ± 0.019 | 13.3 ± 4.2 |

Table 2 shows the maximum muscle contractile force (Emax (g/mg tissue)) and the effective dose for inducing 50% muscle contraction (ED₅₀ (M)) of carbachol-treated mucosal epithelium (−) specimens of tracheal smooth muscles from the STZ-induced diabetic rat model. Data is presented as mean values±standard deviations (n=6).

### Example 2

### Nerve Injury-Preventive Effect on Vascular Smooth Muscles of Streptozotocin-Induced Diabetic Rats

### [Test Method]

Eight-week-old male SD rats were divided into four groups, three of which were intraperitoneally administered with 50 mg/kg of streptozotocin (hereafter, also referred to as STZ) to induce diabetes. Of this diabetic model, two groups were used as the test substance-administered groups, and were intraperitoneally administered with 2 mg/kg or 8 mg/kg of “compound 24” synthesized in Preparation Example 2 once daily for four weeks continuously. The diabetic model which was not administered with “compound 24” was used as the control group, and rats which were administered with neither STZ nor “compound 24” were used as the non-treated group.

After four weeks from the start of administration, thoracic aorta were isolated to form ring specimens (endothelium (+) specimens) of about 2 mm in length. The vascular endothelia were further removed from these specimens to form endothelium (−) specimens. These specimens were suspended in an organ bath, and the contractile response to cumulative administration of 9,11-dideoxy-9α,11α-methanoepoxy-prosta-5Z,13E-dien-1-oic acid (CAS Registry No.: 56985-40-1, hereafter also referred to as U-46619), which is a thromboxane A2 derivative, was observed. The muscle contractile force was measured with various cumulative concentrations of U-46619 within the range of 10^{-9.0} to 10^{-6.5} M, to obtain the maximum muscle contractile force (Emax (g/mg tissue)) and the effective dose for inducing 50% muscle contraction (ED₅₀ (M)).

### [Results]

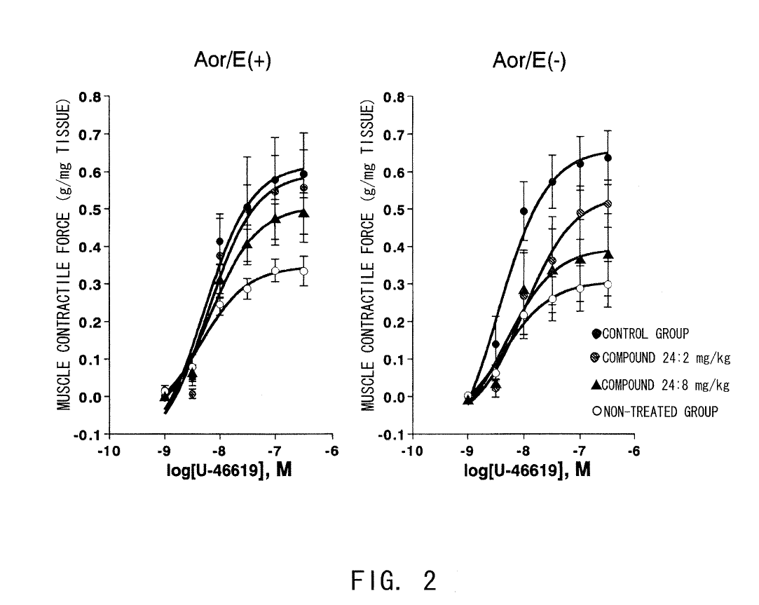

The results are shown in FIG. 2, Table 3, and Table 4. In both endothelium (+) specimens and endothelium (−) specimens, the control group showed greater Emax increase than the non-treated group after vascular smooth muscle contraction was induced with U-46619; however, the administration of “compound 24” inhibited the Emax increase in a dose-dependent manner. This tendency was more remarkable in endothelium (−) specimens, and the control group showed a significant Emax increase compared to the non-treated group. Furthermore, the group administered with 2 mg/kg or 8 mg/kg of “compound 24” showed significant inhibition of the Emax increase compared to the control group. In endothelium (+) specimens, the group administered with 8 mg/kg of “compound 24” showed significant inhibition of the Emax increase compared to the control group. In contrast, there was no difference in ED₅₀ among the groups. From these results, “compound 24” was found to have a vascular smooth muscle contraction-inhibiting effect.

TABLE 3

| | Emax | ED₅₀ |
|---|---|---|
| | (g/mg tissue) | (×10⁻⁹ M) |
| Non-treated group | 0.339 ± 0.038 | 8.218 ± 0.053 |
| Control group | 0.598 ± 0.069 | 8.139 ± 0.036 |
| Compound 24: 2 mg/kg | 0.576 ± 0.082 | 8.077 ± 0.037 |
| Compound 24: 8 mg/kg | 0.416* ± 0.033 | 8.102 ± 0.055 |

Table 3 shows the maximum muscle contractile force (Emax (g/mg tissue)) and the effective dose for inducing 50% muscle contraction (ED₅₀ (M)) of endothelium (+) specimens of U-46619-treated vascular smooth muscles from the STZ-induced diabetic rat model. Data is presented as mean values±standard deviations (n=6).

*: p<0.05 (vs. control group, student's t-test)

TABLE 4

| | Emax | ED₅₀ |
|---|---|---|
| | (g/mg tissue) | (×10⁻⁹ M) |
| Non-treated group | 0.315 ± 0.052 | 8.19 ± 0.074 |
| Control group | 0.639# ± 0.070 | 8.235 ± 0.073 |
| Compound 24: 2 mg/kg | 0.498* ± 0.080 | 8.149 ± 0.030 |
| Compound 24: 8 mg/kg | 0.357* ± 0.054 | 8.194 ± 0.114 |

Table 4 shows the maximum muscle contractile force (Emax (g/mg tissue)) and the effective dose for inducing 50% muscle contraction (ED₅₀ (M)) of endothelium (−) specimens of U-46619-treated vascular smooth muscles from the STZ-induced diabetic rat model. Data is presented as mean values±standard deviations (n=6).

*: p<0.05 (vs. control group, student's t-test)
#: p<0.05 (vs. non-treated group, student's t-test)

### INDUSTRIAL APPLICABILITY

Compound (1) improved vascular and tracheal smooth muscle dysfunctions in a diabetic animal model, and is thus useful as a preventive and/or therapeutic drug for diabetic vascular disorders and respiratory difficulties.

1. A method for either one, or both, of preventing and treating at least one disorder selected from the group consisting of diabetic vascular disorders and diabetic respiratory disorders, comprising the step of administering a cyclohexenone long chain alcohol compound represented by the following formula (1): 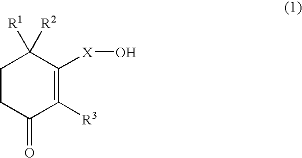
wherein R¹, R², and R³ each represent a hydrogen atom or a methyl group, and X represents a linear or branched C10-C28 alkylene or alkenylene group.

2. The method of claim 1, wherein R¹ is a methyl group and X is a linear C10-C28 alkylene group.

3. The method of claim 2, wherein R² is a methyl group.

4. The method of claim 2, wherein R³ is a methyl group.

5. The method of claim 1, wherein R¹ and R² are hydrogen atoms.

6. The method of claim 1, wherein the cyclohexenone long chain alcohol compound is selected from the group consisting of the following compounds:
3-(10-hydroxydecyl)-2-cyclohexen-1-one (3-(10-hydroxydecyl)-2-cyclohexenone);
3-(1-hydroxyundecyl)-2-cyclohexen-1-one (3-(11-hydroxyundecyl)-2-cyclohexenone);
3-(12-hydroxydodecyl)-2-cyclohexen-1-one (3-(12-hydroxydodecyl)-2-cyclohexenone);
3-(13-hydroxytridecyl)-2-cyclohexen-1-one (3-(13-hydroxytridecyl)-2-cyclohexenone);
3-(14-hydroxytetradecyl)-2-cyclohexen-1-one (3-(14-hydroxytetradecyl)-2-cyclohexenone);
3-(10-hydroxydecyl)-4-methyl-2-cyclohexen-1-one (3-(10-hydroxydecyl)-4-methyl-2-cyclohexenone);
3-(11-hydroxyundecyl)-4-methyl-2-cyclohexen-1-one (3-(11-hydroxyundecyl)-4-methyl-2-cyclohexenone);
3-(12-hydroxydodecyl)-4-methyl-2-cyclohexen-1-one (3-(12-hydroxydodecyl)-4-methyl-2-cyclohexenone);
3-(13-hydroxytridecyl)-4-methyl-2-cyclohexen-1-one (3-(13-hydroxytridecyl)-4-methyl-2-cyclohexenone);
3-(14-hydroxytetradecyl)-4-methyl-2-cyclohexen-1-one (3-(14-hydroxytetradecyl)-4-methyl-2-cyclohexenone);
4,4-dimethyl-3-(10-hydroxydecyl)-2-cyclohexen-1-one (4,4-dimethyl-3-(10-hydroxydecyl)-2-cyclohexenone);
3-(11-hydroxyundecyl)-4,4-dimethyl-2-cyclohexen-1-one (3-(11-hydroxyundecyl)-4,4-dimethyl-2-cyclohexenone);
3-(12-hydroxydodecyl)-4,4-dimethyl-2-cyclohexen-1-one (3-(12-hydroxydodecyl)-4,4-dimethyl-2-cyclohexenone);
3-(13-hydroxytridecyl)-4,4-dimethyl-2-cyclohexen-1-one (3-(13-hydroxytridecyl)-4,4-dimethyl-2-cyclohexenone);
3-(14-hydroxytetradecyl)-4,4-dimethyl-2-cyclohexen-1-one (3-(14-hydroxytetradecyl)-4,4-dimethyl-2-cyclohexenone);
3-(10-hydroxydecyl)-2-methyl-2-cyclohexen-1-one (3-(10-hydroxydecyl)-2-methyl-2-cyclohexenone);
3-(11-hydroxyundecyl)-2-methyl-2-cyclohexen-1-one (3′-(11-hydroxyundecyl)-2-methyl-2-cyclohexenone);
3-(12-hydroxydodecyl)-2-methyl-2-cyclohexen-1-one (3-(12-hydroxydodecyl)-2-methyl-2-cyclohexenone);
3-(13-hydroxytridecyl)-2-methyl-2-cyclohexen-1-one (3-(13-hydroxytridecyl)-2-methyl-2-cyclohexenone);
3-(14-hydroxytetradecyl)-2-methyl-2-cyclohexen-1-one (3-(14-hydroxytetradecyl)-2-methyl-2-cyclohexenone);
3-(12-hydroxydodecyl)-2,4,4-trimethyl-2-cyclohexen-1-one (3-(12-hydroxydodecyl)-2,4,4-trimethyl-2-cyclohexenone);
3-(13-hydroxytridecyl)-2,4,4-trimethyl-2-cyclohexen-1-one (3-(13-hydroxydodecyl)-2,4,4-trimethyl-2-cyclohexenone);
3-(14-hydroxytetradecyl)-2,4,4-trimethyl-2-cyclohexen-1-one (3-(14-hydroxytetradecyl)-2,4,4-trimethyl-2-cyclohexenone);
3-(15-hydroxypentadecyl)-2,4,4-trimethyl-2-cyclohexen-1-one (3-(15-hydroxypentadecyl)-2,4,4-trimethyl-2-cyclohexenone); and
3-(16-hydroxyhexadecyl)-2,4,4-trimethyl-2-cyclohexen-1-one (3-(16-hydroxyhexadecyl)-2,4,4-trimethyl-2-cyclohexenone).

7. The method of claim 1, wherein the disorder is a diabetic vascular disorder.

8. The method of claim 1, wherein the disorder is a diabetic respiratory disorder.

9. The method of claim 8, wherein the diabetic respiratory disorder is sleep apnea syndrome.

10. The method of claim 1, wherein the method is a therapeutic method.

11. A preventive and/or therapeutic agent for either one, or both, of a diabetic vascular disorder and a diabetic respiratory disorder, comprising, as an active ingredient, a cyclohexenone long chain alcohol compound represented by the following formula (1): 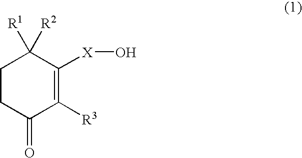
wherein R¹, R², and R³ each represent a hydrogen atom or a methyl group, and X represents a linear or branched C10-C28 alkylene or alkenylene group.

12. The method of claim 3, wherein R³ is a methyl group.
